(19) United States
(12) Patent Application Publication
(10) Pub. No.: US 2021/0275719 A1
(43) Pub. Date: Sep. 9, 2021

(54) EXTERNAL DRESSING AS WELL AS PREPARATION METHOD AND APPLICATION THEREOF

(71) Applicant: Jun Cai, WUHAN (CN)

(72) Inventor: Jun CAI, WUHAN (CN)

(21) Appl. No.: 16/838,813

(22) Filed: Apr. 2, 2020

(30) Foreign Application Priority Data
Mar. 9, 2020 (CN) 202010158211.3

(51) Int. Cl. A61L 26/00

(52) U.S. Cl. CPC A61L 26/0023; A61L 26/0066; A61L 2300/252; A61L 2300/42; A61L 26/0033

(57) ABSTRACT

The present invention relates to an external dressing as well as a preparation method and application thereof. The external dressing includes: A. a solvent, which is a mixture including normal saline and an HP value regulator, B. a natural polymer as a thickener; and C. urokinase, the content of which is 0.05-50,000 U/ml. Experiments in mice prove that the present invention has obvious curative effect on wound rehabilitation, and has the advantages of easy realization and operation, fewer side effects and benefit for popularization and application.

### CROSS-REFERENCE TO RELATED APPLICATIONS

This application claims priority to Chinese Patent Application No. 202010158211.3 with a filing date of Mar. 9, 2020. The content of the aforementioned applications, including any intervening amendments thereto, are incorporated herein by reference.

### TECHNICAL FIELD

The present invention relates to the medical field, in particular to an external dressing capable of promoting wound rehabilitation. The liquid dressing not only can clean the wound through enzymology, but also can effectively promote angiogenesis of the wound and accelerate the healing process. The present invention also provides a preparation method of the external dressing and application of the external dressing in skin wound therapy.

### BACKGROUND OF THE PRESENT INVENTION

Skin wound healing includes key steps of filling with new granulation tissues and covering with wound scar tissues after fibroblast differentiation. Current relevant researches show that for some common traumatic wounds, the normal dressing is used to maintain appropriate humidity of wounds and drainage of local necrotic secretions so that the wounds can be rehabilitated. However, for some chronic wounds difficult to heal, such as foot skin wounds of diabetic patients and skin pressure sore wounds of long-term bedridden patients, the wound healing course is longer and most of the wounds have poor prognosis after simply changing the dressing and covering with ordinary gauze dressing. The foot skin wounds of some diabetic patients fail to heal clinically; and amputation is finally selected to control ulcer enlargement and repeated infection of ulcer surfaces.

Blood supply disorder in the local injury region after skin trauma is considered as one of main causes of difficult natural healing of skin wounds, especially in patients with blood supply disorder (such as diabetic patients and elderly bedridden patients). Stimulating the regeneration of capillaries in the skin injury region and reconstructing the blood supply of skin dermis are focal points for promoting the healing of skin wounds. In recent years, a method of dropwise adding relevant extracellular factors (biological agents) and/or drugs into the local wounds is used clinically. The measures have indeed promoted angiogenesis and played a role of promoting wound healing of injured sites to a certain extent, but have apparent shortcomings. Firstly, the utilization rate of the relevant drugs and extracellular growth factors (biological agents) is low; and the dose of drugs actually reaching the injured sites is unknown. Secondly, a method of local injection may cause new complications of other tissues and organs, is too high in risk, and may cause pain, irritation, potential infection, tumorigenic risks and the like of affected parts.

At present, for the skin wounds of the diabetic patients, more and more emphasis is placed on the local therapy of skin injury while systematically controlling the blood glucose level; and a negative pressure wound therapy (NPWT) at wound sites is one of the commonly used methods for local therapy of refractory acute and chronic wounds. The NPWT can promote wound healing and show clinical efficacy by reducing exudate and tissue edema in wound region, inhibiting bacterial growth and performing mechanical emergency reaction to induce granulation tissue formation and angiogenesis. However, with the increase of therapeutic indications, some consequent new problems, such as the optimal negative pressure value and the dressing change time need further study. In addition, novel dressings, such as artificial skin, are constantly developed as temporary skin substitutes for chronic wounds. Consequently, people try to add some cytokines (such as vascular endothelial growth factors (VEGF), platelet-derived growth factors (PDGF) and stromal cell-derived factors (SDF)-1α) with an angiogenesis inducing effect into the dressings prepared from biomaterials.

Skin wound healing is a continuous biological cascade reaction process. An initial process of skin healing can be roughly divided into three stages according to histological advantages of different time phases as follows: a first stage: 1-3 days after forming the wound, it is mainly manifested as inflammatory reaction in the skin wound region, accompanied by serous exudation and hyperemia and neutrophil-dominated leukocyte infiltration; a second stage: 3-14 days after forming the wound, it is manifested as apparent granulation tissue hyperplasia, angiogenesis, microvascular system reconstruction, collagen formation and gradual filling of the wound with new connective tissues; and a third stage: 3-21 days after forming the wound, it is manifested as assisted coverage of surfaces of granulation tissues with simple epithelium composed of basal cells, collagen recombination and the like. Therefore, the wound dressing should have characteristics that blood vessel promoting drugs should be rapidly released at an early stage to collect cells and vascular formation-related cells and factors to reach the injured region and rapidly clean necrotic tissues of the wound to provide a basis for the upcoming vascular reconstruction. An external dressing capable of meeting the above requirements needs to be developed in the prior art.

### SUMMARY OF PRESENT INVENTION

The technical problem to be solved by the present invention is to provide a urokinase-containing external dressing and a method for preparing the external dressing, so as to treat skin trauma more effectively and rapidly.

The external dressing is characterized by including:

A. a solvent, wherein the solvent contains a sodium chloride solution, and the content of sodium chloride is 0.9% of the total weight of the external dressing;

B. a natural polymer, which includes any one or more of the following components: chitosan, cellulose, hyaluronic acid, collagen and sodium alginate,

wherein the molecular weight of chitosan is 40,000-70,000; the weight ratio of the chitosan to the finished external dressing is 5%2%; the weight ratio of the cellulose to the solvent is not greater than 5%; the weight ratio of the hyaluronic acid to the solvent is not greater than 2%; the weight ratio of the collagen to the solvent is not greater than 0.05%; the weight ratio of the gelatin to the solvent is not greater than 5%; and the weight ratio of the sodium alginate to the solvent is not greater than 2%; and

C. urokinase, the content of which is 0.05-50,000 U/ml.

In one embodiment, the solvent also contains an HP value regulator; the content of the HP value regulator is not greater than 0.3% of the total weight of liquid; the HP value regulator is hydrogen phosphate and/or bicarbonate; and the PH value of the external dressing ranges from 7.0 to 7.5.

Preferably, the content of the urokinase in the solvent is 0.99-10,100 U/ml.

More preferably, the content of the urokinase in the solvent is 10,000 U/ml.

A preparation method of the external dressing is characterized by including the following steps:

(i) preparing sodium chloride and pure water, and mixing uniformly into normal saline to form a solvent;

(ii) preparing a natural polymer, wherein the natural polymer is selected from one or more of chitosan, cellulose, hyaluronic acid, collagen, gelatin and sodium alginate; mixing the selected natural polymer with the solvent to meet the following conditions: if the chitosan is present in the selected natural polymer, the concentration of the chitosan is a weight ratio 5%±2% of the chitosan to the finished external dressing; if the cellulose is present, the weight ratio of the cellulose to the solvent is not greater than 5%; if the hyaluronic acid is present, the weight ratio of the hyaluronic acid to the solvent is not greater than 2%; if the collagen is present, the weight ratio of the collagen to the solvent is not greater than 0.05%; if the gelatin is present, the weight ratio of the gelatin to the solvent is not greater than 5%; and if the sodium alginate is present, the weight ratio of the sodium alginate to the solvent is not greater than 2%;

(iii) forming a liquid dressing containing urokinase by the natural polymer selected in the solvent and the urokinase, wherein the content of the urokinase in the liquid dressing is 0.05-50,000 U/ml; regulating and adding sodium chloride to account for 0.9%/c by weight of the finished product; and integrally stirring and mixing into a colorless or white translucent gelatinous substance.

Preferably, hydrogen phosphate and/or bicarbonate is mixed in the mixed solvent; and the total content of the hydrogen phosphate and/or bicarbonate is not greater than 0.3% of the total weight of the external dressing, so that the PH value of the finally formed external dressing is 7.0-7.5.

Preferably, the content of the urokinase in the external dressing is 0.5-20,000 U/ml.

More preferably, the content of the urokinase in the external dressing is 10,000 U/ml.

For application of the urokinase-containing external dressing, the external dressing contains a solvent and the urokinase; the solvent of the finished external dressing contains sodium chloride accounting for 0.9% by weight of the external dressing; the content of the urokinase in the finished external dressing is 0.05-50,000 U/ml; and the external dressing is applied to therapy of skin wounds.

The skin wounds include common trauma wounds, burn wounds, refractory wounds, diabetic wounds and pressure sore wounds.

Further, the external dressing contains a natural polymer which contains any one or more of the following components: chitosan, cellulose, hyaluronic acid, collagen and sodium alginate,

wherein the molecular weight of the chitosan is 40,000-70,000; the weight ratio of the chitosan to the solvent is 5%±2%; the weight ratio of the cellulose to the solvent is not greater than 5%; the weight ratio of the hyaluronic acid to the solvent is not greater than 2%; the weight ratio of the collagen to the solvent is not greater than 0.05%; the weight ratio of the gelatin to the solvent is not greater than 5%; and the weight ratio of the sodium alginate to the solvent is not greater than 2%.

If the external dressing contains two or more different natural polymers, the weight ratios of the components to the solvent are all not greater than the above values.

As an embodiment, the solvent also contains an HP value regulator, the content of the HP value regulator is not greater than 0.3% of the total weight of the external dressing; and the HP value regulator is hydrogen phosphate and/or bicarbonate, so that the PH value of the external dressing ranges from 7.0 to 7.5.

Urokinase, as an activating enzyme of plasminogen, activates wound plasmase and plays a role of liquefying and draining necrotic tissues and bacterial metabolites of the wounds. Urokinase-type plasminogen activator (uPA) is considered to play a key role of vascular remodeling after injury, and can also mediate and regulate cell migration in vascular walls. The uPA is also considered to play a key role of mediating homing, proliferation and differentiation of mesenchymal stem cells in inflammation and wound healing. The urokinase is directly applied to the wounds, so that the tumorigenesis induction of chronic inflammatory tissues due to direct application of various cell growth factors is also avoided.

Experiments in mice further prove that the effective and optimal content of the urokinase is determined by the experiments in the present invention, and the prepared urokinase-containing gelatinous external dressing convenient for clinical application not only has convenient use, but also has obvious efficacy on wound healing and has the advantages of easy realization and operation, fewer side effects and benefit for popularization.

### DESCRIPTION OF THE DRAWINGS

FIG. 1 is a numerical distribution diagram of urokinase with different concentrations on chronic wound healing area in mice and shows that the concentration 10000 U/ml (10000 units per ml) is the optimal concentration for wound healing;

FIG. 2 is a curve of influence of chitosan normal saline containing 10,000 U/ml of urokinase (series 2) on healing of clean wounds of healthy Kunming mice in 8 days, wherein the solid line refers to a normal saline control group (series 1); and

FIG. 3 is a curve of influence of chitosan normal saline containing 10,000 U/ml of urokinase (series 2) on wound healing of diabetic mice in 8 days, wherein the solid line refers to a chitosan normal saline control group (series 1).

### DETAILED DESCRIPTION OF PREFERRED EMBODIMENTS

The present invention will be further described with reference to embodiments below. An external dressing of the present invention is a urokinase-containing external dressing, which includes a solvent, a natural polymer and urokinase, wherein normal saline is used as a basic solvent; one or a mixture of chitosan, cellulose, hyaluronic acid, collagen and sodium alginate is used as the natural polymer; and the natural polymer is used as a thickener so that the final product is gelatinous. In the raw materials that can be used as the natural polymer, the molecular weight of the chitosan is 40,000-70,000, and the weight ratio of the chitosan to the solvent is 5%±2%, i.e., the weight ratio of the chitosan to the solvent is 3%-7%; the weight ratio of the cellulose to the solvent is not greater than 5%, i.e., the weight ratio of the cellulose to the solvent is 0-5%; the weight ratio of the hyaluronic acid to the solvent is not greater than 2%, i.e., the weight ratio of the hyaluronic acid to the solvent is 0-2%; the weight ratio of the collagen to the solvent is not greater than 0.05%, i.e., the weight ratio of the collagen to the solvent is 0-0.05%; the weight ratio of the gelatin to the solvent is not greater than 5%, i.e., the weight ratio of the gelatin to the solvent is 0-5%; and the weight ratio of the sodium alginate to the solvent is not greater than 2%, i.e., the weight ratio of the sodium alginate to the solvent is 0-2%. If the external dressing contains two or more different natural polymers, the weight ratios of the components to the solvent are not greater than the above values. A preparation method of the external dressing includes a step of mixing the urokinase with a solution containing the natural polymer. The content of the urokinase is 0.05-50,000 U/ml. The urokinase is used for preparing dressing products for treating skin trauma. Skin wounds include common trauma wounds, burn wounds, refractory wounds, diabetic wounds and pressure sore wounds.

In the solvent, sodium chloride as a raw material shall meet the chemical analytical purity requirement of the content ≥99.5%; and sodium chloride and pure water are mixed into normal saline with the weight ratio of 0.9% as the solvent.

The natural polymer may include one or more of chitosan, cellulose, hyaluronic acid, collagen, gelatin and sodium alginate; and the selected raw materials shall meet the analytical purity requirement of the content ≥99.5%. Any one of natural polymers such as chitosan, hyaluronic acid, collagen and sodium alginate shall be dissolved within a temperature range of 30° C.±5° C., and shall be fully dissolved by stirring with a stirrer at 1000±500 r/min (revolutions per minute) for 30±5 min, wherein the cellulose shall be fully dissolved by heating and gelatinizing at 95° C. and stirring with the stirrer at 100

To adjust the HP value, analytically pure hydrogen phosphate and/or bicarbonate with the content ≥99.5% is added into the mixture at 0.3% of the total weight of the external dressing so that the PH value of the external dressing ranges from 7.0 to 7.5.

The natural polymer and the urokinase are uniformly mixed in the solvent, heated at 30° C.±5° C., and fully dissolved 1000±500 r/min for 10 min; the dissolved mixture shall be a colorless or slightly white translucent gelatinous substance. Thus, a urokinase-containing liquid dressing is formed.

As an embodiment, the composition of the external dressing is 0.9% by weight of normal saline as the solvent, wherein sodium chloride as the raw material shall meet the chemical analytical purity requirement of the content ≥99.5%; and sodium chloride and pure water are mixed into the normal saline as the solvent. The natural polymer, 5% by weight of chitosan, having the molecular weight of 40,000-70,000 is mixed with the solvent; the chitosan raw material shall meet the analytical purity requirement of content ≥99.5%; and the chitosan shall be fully dissolved by stirring with the stirrer at 1000 r/min at 30° C. or 30 min. Urokinase is mixed uniformly. In the application of the urokinase-containing external dressing, the concentration of urokinase is tested in groups as follows; and each group data are used as different embodiments to provide experimental data for comparison of proportions and effects.

1. Test Materials and Methods:

1.1 Laboratory Animals and Reagents

50 SPF (Specific Pathogen Free) male Kunning mice with a body weight of about 30 g were provided by the Laboratory Animal Center of Tongji Medical College, Huazhong University of Science and Technology. Chloral hydrate and carboxymethyl chitosan lyophilized powder were purchased from Sigma Company. Urokinase lyophilized powder was purchased from Nanjing Nanda Pharmaceutical Co., Ltd.

1.2 Establishment of Chronic Wound Model for Diabetic Mice and Grouping Administration Method

1.2.1 Establishment of Chronic Wound Model for Diabetic Mice

The mice were labeled and weighed and then intraperitoneally injected with STZ (streptozotocin) (50 mg/kg) continuously for four days after labelling and weighing. After the fourth injection, the mice were fasted without water deprivation for 10 h (hours). Blood was taken from the caudal vein for measuring the fasting blood glucose level by a rapid blood glucose meter. The mice having a fasting blood glucose concentration of greater than 11.1 mmol/L and continuous polydipsia, polyphagia and polyuria were selected as diabetic mice. After the model was established for eight weeks, the mice were anesthetized by intraperitoneal injection of 10% chloral hydrate (30 mg/kg), were perforated on both sides of the midlines of the backs (spacing more than 1 cm) with a perforator having the length and width of 1 cm, were wrapped with sterile gauzes after hemostasis and were fed in a single cage.

1.2.2 Experimental Grouping

A total of five experimental groups, including a group of diabetic chronic wound mice treated with urokinase at different concentrations, a group of healthy mice treated with urokinase, a group of healthy mice subjected to conventional therapy, a group of diabetic chronic wound treated with urokinase and a group of diabetic wound mice subjected to conventional therapy, were provided, wherein each group included 9 mice.

1.2.3 Administration Method

After the wounds on the backs of the mice were successfully established, 5% chitosan normal saline and carboxymethyl chitosan saline external dressing with different urokinase concentrations were respectively applied to the wounds once a day for eight days.

1.3 Test Items

1.3.1 Specimen Collection

On the 1st, 2nd, 3rd, 4th, 5th, 6th, 7th and 8th days after therapy, a digital camera was used to photograph the wounds and record the healed areas. Computer image analysis software was used to calculate the areas and evaluate the wound healing effect.

1.4 Statistical Methods

All measurement data were expressed as X̅±S (indicating a mean t a standard deviation); and SPSS 10.0 software was used for T-test (student test).

2 Results

2.1 Observation on Chronic Wound Healing of Diabetic Mice Treated with Carboxymethyl Chitosan Urokinase Saline External Dressings with Different Urokinase Concentrations.

The results of comparing and recording the healing areas of the chronic wounds of eight diabetic experimental mice after 5 days of treatment with the carboxymethyl chitosan urokinase saline external dressing with different urokinase concentrations are as follows:

| | Concentration | Concentration | Concentration | Concentration | Concenration | Concentration | Concentration |
|---|---|---|---|---|---|---|---|
| | A | B | C | D | E | F | G |
| | l000 u/ml | 5000 u/ml | 10000 u/ml | 20000 u/ml | 40000 u/ml | 80000 u/ml | 160000 u/ml |
| Healing aera | 0.34 | 0.57 | 0.82 | 0.83 | 0.76 | 0.56 | 0.75 |
| Healing aera | 0.35 | 0.5 | 0.86 | 0.76 | 0.83 | 0.88 | 0.85 |
| Healing aera | 0.34 | 0.64 | 0.76 | 0.56 | 0.75 | 0.82 | 0.56 |
| Healing aera | 0.62 | 0.34 | 0.59 | 0.88 | 0.72 | 0.86 | 0.88 |
| Healing aera | 0.32 | 0.62 | 0.88 | 0.75 | 0.52 | 0.56 | 0.76 |
| Healing aera | 0.34 | 0.56 | 0.75 | 0.69 | 0.69 | 0.88 | 0.83 |
| Healing aera | 0.21 | 0.66 | 0.85 | 0.85 | 0.72 | 0.82 | 0.85 |
| Healing aera | 0.29 | 0.51 | 0.76 | 0.88 | 0.88 | 0.86 | 0.76 |
| Healing aera | 0.28 | 0.63 | 0.67 | 0.74 | 0.68 | 0.75 | 0.82 |
| Mean | 0.343333 | 0.558889 | 0.771111 | 0.771111 | 0.727778 | 0.776667 | 0.784444 |
| Standard | 0.112805 | 0.093979 | 0.094927 | 0.103736 | 0.101338 | 0.129228 | 0.096061 |
| deviation | | | | | | | |

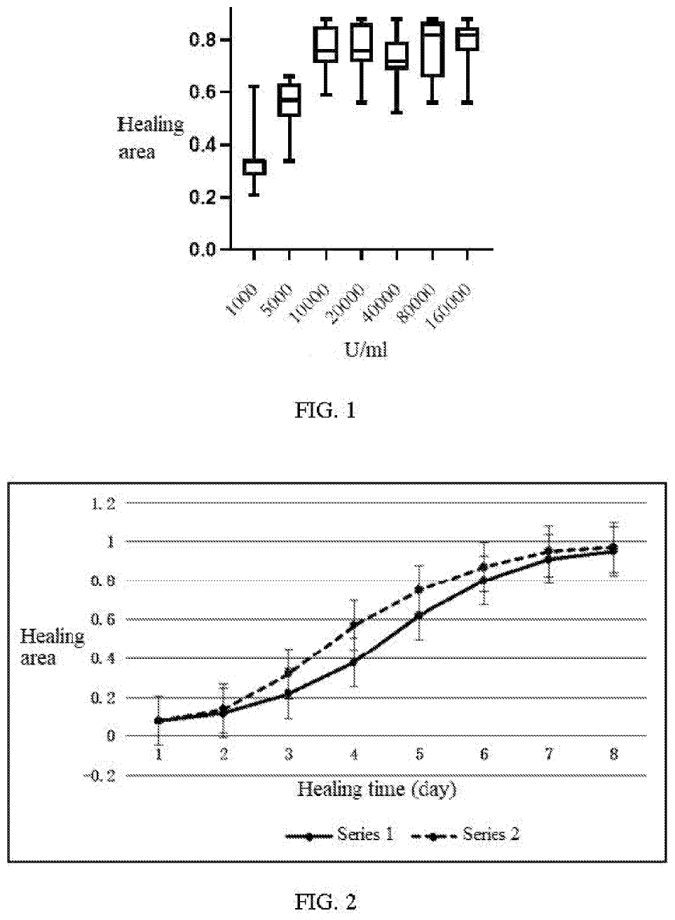

Numerical distribution diagram of urokinase with different concentrations on chronic wound healing area in mice is shown in FIG. 1 and shows that the concentration 10,000 U/ml (10,000 units per ml) is the optimal concentration for wound healing.

After observing the chronic wounds of nine diabetic mice treated with carboxymethyl chitosan saline external dressings with different urokinase concentrations for 5 days, the wound healing records show that the wounds represent the optimal wound healing ability at a urokinase concentration of 10,000 U/ml.

2.2 Influence of Solution Containing 10,000 U/ml of Urokinase on Clean Wound Healing of Healthy Kunming Mice

After the back wounds of healthy mice were successfully established, carboxymethyl chitosan normal saline (group C) and 10,000 U/ml urokinase (group T) were applied to the wounds once a day (DAY1-DAY8) according to different groups. The wound recovery areas were measured before changing the dressings every day. The experimental records were as follows:

Influence of the carboxymethyl chitosan saline external dressing containing 10,000 U/ml of urokinase on healing of clean wounds of healthy Kunming mice in 1st-4th days:

| | C-DAY 1 | T-DAY 1 | C-DAY 2 | T-DAY 2 | C-DAY 3 | T-DAY 3 | C-DAY 4 | T-DAY 4 |
|---|---|---|---|---|---|---|---|---|
| 1# | 0.08 | 0.06 | 0.12 | 0.14 | 0.22 | 0.32 | 0.38 | 0.57 |
| 2# | 0.07 | 0.08 | 0.09 | 0.12 | 0.2 | 0.3 | 0.34 | 0.61 |
| 3# | 0.08 | 0.09 | 0.12 | 0.14 | 0.22 | 0.32 | 0.38 | 0.54 |
| 4# | 0.09 | 0.08 | 0.14 | 0.15 | 0.25 | 0.35 | 0.42 | 0.57 |
| 5# | 0.08 | 0.09 | 0.12 | 0.14 | 0.22 | 0.32 | 0.36 | 0.6 |
| 6# | 0.07 | 0.08 | 0.11 | 0.12 | 0.24 | 0.28 | 0.38 | 0.52 |
| 7# | 0.08 | 0.07 | 0.12 | 0.14 | 0.22 | 0.32 | 0.42 | 0.57 |
| 8# | 0.09 | 0.09 | 0.13 | 0.16 | 0.2 | 0.38 | 0.37 | 0.55 |
| Mean | 0.08 | 0.08 | 0.1186 | 0.1388 | 0.2213 | 0.3238 | 0.3813 | 0.5663 |
| Standard | 0.0071 | 0.0107 | 0.0146 | 0.0136 | 0.0173 | 0.0302 | 0.0275 | 0.0297 |
| deviation | | | | | | | | |

| | | | | |
|---|---|---|---|---|
| P value | >0.99 | 0.0131 | <0.0001 | <0.0001 |

Influence of carboxymethyl chitosan saline external dressing containing 10,000 U/ml of urokinase on healing of clean wounds of the healthy Kunming mice in 5th-8th days;

| | C-DAY 5 | T-DAY 5 | C-DAY 6 | T-DAY 6 | C-DAY 7 | T-DAY 7 | C-DAY 8 | T-DAY 8 |
|---|---|---|---|---|---|---|---|---|
| 1# | 0.62 | 0.75 | 0.8 | 0.87 | 0.91 | 0.95 | 0.95 | 0.97 |
| 2# | 0.66 | 0.8 | 0.81 | 0.93 | 0.95 | 0.92 | 0.93 | 0.96 |
| 3# | 0.62 | 0.71 | 0.76 | 0.82 | 0.91 | 0.97 | 0.95 | 0.98 |
| 4# | 0.57 | 0.75 | 0.81 | 0.87 | 0.86 | 0.95 | 0.93 | 0.97 |
| 5# | 0.59 | 0.82 | 0.78 | 0.92 | 0.93 | 0.93 | 0.95 | 0.98 |
| 6# | 0.62 | 0.7 | 0.82 | 0.84 | 0.91 | 0.98 | 0.96 | 0.96 |
| 7# | 0.63 | 0.75 | 0.79 | 0.8 | 0.86 | 0.95 | 0.97 | 0.97 |
| 8# | 0.61 | 0.72 | 0.84 | 0.93 | 0.92 | 0.96 | 0.95 | 0.96 |
| Mean | 0.615 | 0.75 | 0.8013 | 0.8725 | 0.9063 | 0.9513 | 0.9488 | 0.9688 |
| Standard | 0.0267 | 0.0421 | 0.0247 | 0.0506 | 0.0316 | 0.0196 | 0.0136 | 0.0083 |
| deviation | | | | | | | | |

| | | | | |
|---|---|---|---|---|
| P value | <0.0001 | 0.003 | 0.0041 | 0.0032 |

The growth curve of clean wounds treated with urokinase-containing carboxymethyl chitosan saline external dressing is shown in FIG. 2.

FIG. 2 shows the influence of chitosan normal saline external dressing 10,000 U/ml of urokinase (series 2) on healing of clean wounds of healthy Kunming mice in 8 days.

The above data show that the wound healing rate of the group of mice treated with urokinase is obviously higher than that of the control group from the 3rd day after wound formation (P<0.01).

2.3 Influence of Carboxymethyl Chitosan Saline External Dressing Containing 10,000 U/ml of Urokinase on Wound Healing of the Chronic Diabetic Mice

After the back wounds of diabetic mice were successfully established, normal saline chitosan (group C) and 10,000 U/ml urokinase carboxymethyl chitosan (group T) were respectively applied to the wounds once a day for continuous 8 days according to different groups. The wound recovery areas were measured before changing the dressings every day.

The following table shows the influence of the carboxymethyl chitosan saline external dressing containing 10,000 U/ml of urokinase on healing of wounds of the diabetic mice in 1st-4th days:

| | C-DAY 1 | T-DAY 1 | C-DAY 2 | T-DAY 2 | C-DAY 3 | T-DAY 3 | C-DAY 4 | T-DAY 4 |
|---|---|---|---|---|---|---|---|---|
| 1# | 0.08 | 0.1 | 0.11 | 0.17 | 0.2 | 0.31 | 0.37 | 0.55 |
| 2# | 0.07 | 0.09 | 0.13 | 0.15 | 0.23 | 0.34 | 0.35 | 0.56 |
| 3# | 0.08 | 0.11 | 0.09 | 0.19 | 0.17 | 0.28 | 0.38 | 0.58 |
| 4# | 0.09 | 0.12 | 0.12 | 0.16 | 0.24 | 0.31 | 0.38 | 0.53 |
| 5# | 0.07 | 0.08 | 0.11 | 0.18 | 0.17 | 0.27 | 0.36 | 0.52 |
| 6# | 0.08 | 0.07 | 0.08 | 0.18 | 0.19 | 0.31 | 0.34 | 0.55 |
| 7# | 0.08 | 0.13 | 0.09 | 0.14 | 0.23 | 0.33 | 0.38 | 0.54 |
| 8# | 0.08 | 0.07 | 0.12 | 0.15 | 0.18 | 0.29 | 0.36 | 0.54 |
| Mean | 0.0786 | 0.0963 | 0.1063 | 0.165 | 0.2013 | 0.305 | 0.365 | 0.5463 |
| Standard | 0.0065 | 0.0226 | 0.0177 | 0.0177 | 0.0286 | 0.0239 | 0.0151 | 0.0185 |
| deviation | | | | | | | | |

| | | | | |
|---|---|---|---|---|
| P value | 0.054 | <0.0001 | <0.0001 | <0.0001 |

The following table shows the influence of the carboxymethyl chitosan saline external dressing containing 10,000 U/ml of urokinase on healing of clean wounds of the diabetic mice in 4th-8th days:

| | C-DAY 5 | T-DAY 5 | C-DAY 6 | T-DAY 6 | C-DAY 7 | T-DAY 7 | C-DAY 8 | T-DAY 8 |
|---|---|---|---|---|---|---|---|---|
| 1# | 0.53 | 0.71 | 0.63 | 0.84 | 0.68 | 0.93 | 0.71 | 0.96 |
| 2# | 0.52 | 0.73 | 0.65 | 0.82 | 0.71 | 0.91 | 0.68 | 0.98 |
| 3# | 0.55 | 0.66 | 0.61 | 0.85 | 0.66 | 0.91 | 0.69 | 0.95 |
| 4# | 0.48 | 0.74 | 0.59 | 0.84 | 0.66 | 0.95 | 0.73 | 0.97 |
| 5# | 0.56 | 0.73 | 0.62 | 0.84 | 0.73 | 0.92 | 0.72 | 0.94 |
| 6# | 0.48 | 0.69 | 0.64 | 0.79 | 0.72 | 0.93 | 0.67 | 0.95 |
| 7# | 0.57 | 0.73 | 0.62 | 0.86 | 0.65 | 0.91 | 0.71 | 0.96 |
| 8# | 0.54 | 0.66 | 0.64 | 0.86 | 0.66 | 0.94 | 0.73 | 0.94 |
| Mean | 0.5288 | 0.7062 | 0.625 | 0.8375 | 0.6838 | 0.925 | 0.705 | 0.9563 |
| Standard | 0.034 | 0.0324 | 0.0193 | 0.0231 | 0.0316 | 0.0151 | 0.0227 | 0.0131 |
| deviation | | | | | | | | |

| | | | | |
|---|---|---|---|---|
| P value | <0.0001 | <0.0001 | <0.0001 | <0.0001 |

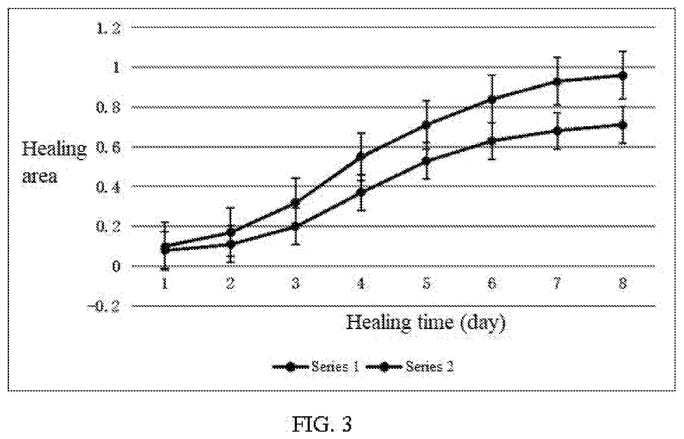

The growth curve of chronic wounds treated with urokinase-containing carboxymethyl chitosan saline external dressing is shown in FIG. 3.

The above data show that the wound healing rate of the group of mice treated with the urokinase-containing carboxymethyl chitosan saline external dressing is obviously higher than that of the control group from the 2nd day after wound formation; and the healing area of the urokinase-containing carboxymethyl chitosan group is obviously and statistically different from that of the control group on the 2nd-7th days (P<0.0001).

In another embodiment, hydrogen phosphate and/or bicarbonate not greater than 0.3% of the total weight of the external dressing can be added into the solvent as the HP value regulator after mixing the natural polymer; the hydrogen phosphate and/or bicarbonate are powdery; and the PH value of the solvent is regulated to 7.0-7.5.

In the natural polymer, any one or more of the following mixed thickeners can be added: cellulose with the weight ratio of not greater than 5% to the solvent, hyaluronic acid with the weight ratio of not greater than 2% to the solvent, collagen with the weight ratio of not greater than 0.05% to the solvent, gelatin with the weight ratio of not greater than 5% to the solvent, and sodium alginate with the weight ratio of not greater than 2% to the solvent, thereby stabilizing the liquid into a transparent or translucent gelatinous product. The solvent, the natural polymer and the urokinase can be mixed out of sequence to prepare the external dressing.

We claim:

1. An external dressing, comprising:
A. a solvent, wherein the solvent contains a sodium chloride solution, and the content of sodium chloride is 0.9% of the total weight of the external dressing;
B. a natural polymer, which comprises any one or more of the following components: chitosan, cellulose, hyaluronic acid, collagen and sodium alginate,
wherein the molecular weight of chitosan is 40,000-70,000; the weight ratio of the chitosan to the finished external dressing is 5%±2%; the weight ratio of the cellulose to the solvent is not greater than 5%; the weight ratio of the hyaluronic acid to the solvent is not greater than 2%; the weight ratio of the collagen to the solvent is not greater than 0.05%; the weight ratio of the gelatin to the solvent is not greater than 5%; and the weight ratio of the sodium alginate to the solvent is not greater than 2%; and
C. urokinase, the content of which is 0.05-50,000 U/ml.

2. The external dressing according to claim 1, wherein the solvent also contains an HP value regulator; the content of the HP value regulator is not greater than 0.3% of the total weight of the external dressing; the HP value regulator is hydrogen phosphate and/or bicarbonate; and the PH value of the external dressing ranges from 7.0 to 7.5.

3. The external dressing according to claim 1, wherein the content of the urokinase in the solvent is 0.99-10,100 U/ml.

4. The external dressing according to claim 3, wherein the content of the urokinase in the solvent is 10,000 U/ml.

5. A preparation method of the external dressing of claim 1, comprising the following steps:
(i) preparing sodium chloride and pure water, and mixing uniformly into normal saline to form a solvent;
(ii) preparing a natural polymer, wherein the natural polymer is selected from one or more of chitosan, cellulose, hyaluronic acid, collagen, gelatin and sodium alginate; mixing the selected natural polymer with the solvent to meet the following conditions: if the chitosan is present in the selected natural polymer, the concentration of the chitosan is a weight ratio 5%±2% of the chitosan to the finished external dressing; if the cellulose is present, the weight ratio of the cellulose to the solvent is not greater than 5%; if the hyaluronic acid is present, the weight ratio of the hyaluronic acid to the solvent is not greater than 2%; if the collagen is present, the weight ratio of the collagen to the solvent is not greater than 0.05%; if the gelatin is present, the weight ratio of the gelatin to the solvent is not greater than 5%; and if the sodium alginate is present, the weight ratio of the sodium alginate to the solvent is not greater than 2%;
(iii) forming a liquid dressing containing urokinase by the natural polymer selected in the solvent and the urokinase, wherein the content of the urokinase in the liquid dressing is 0.05-50,000 U/ml; regulating and adding sodium chloride to account for 0.9% by weight of the finished product; and integrally stirring and mixing into a colorless or white translucent gelatinous substance.

6. The preparation method of the external dressing according to claim 5, wherein the hydrogen phosphate and/or bicarbonate is mixed in the mixed solvent; and the total content of the hydrogen phosphate and/or bicarbonate is not greater than 0.3% of the total weight of the external dressing, so that the PH value of the finally formed external dressing is 7.0-7.5.

7. The preparation method of the external dressing according to claim 6, wherein the content of the urokinase in the external dressing is 0.5-20,000 U/ml.

8. An application of the urokinase-containing external dressing, the external dressing containing a solvent and the urokinase, wherein the solvent of the finished external dressing contains sodium chloride accounting for 0.9% by weight of the external dressing; the content of the urokinase in the finished external dressing is 0.05-50,000 U/ml; the external dressing is applied to therapy of skin wounds; and the skin wounds comprise common trauma wounds, burn wounds, refractory wounds, diabetic wounds and pressure sore wounds.

9. The application of the urokinase-containing external dressing according to claim 8, wherein the external dressing contains a natural polymer which contains any one or more of the following components: chitosan, cellulose, hyaluronic acid, collagen and sodium alginate,
wherein the molecular weight of the chitosan is 40,000-70,000; the weight ratio of the chitosan to the solvent is 5%±2%; the weight ratio of the cellulose to the solvent is not greater than 5%; the weight ratio of the hyaluronic acid to the solvent is not greater than 2%; the weight ratio of the collagen to the solvent is not greater than 0.05%; the weight ratio of the gelatin to the solvent is not greater than 5%; and the weight ratio of the sodium alginate to the solvent is not greater than 2%.

10. The application of the urokinase-containing external dressing according to claim 8, wherein the solvent also contains an HP value regulator, the content of the HP value regulator is not greater than 0.3% of the total weight of the external dressing; and the HP value regulator is hydrogen phosphate and/or bicarbonate, so that the PH value of the external dressing ranges from 7.0 to 7.5.
